(19) United States
(12) Patent Application Publication
(10) Pub. No.: US 2024/0032981 A1
(43) Pub. Date: Feb. 1, 2024

(54) FLUID DELIVERY SYSTEM AND RELATED METHODS OF USE

(71) Applicant: The Pann State Research Foundation, University Park, PA (US)

(72) Inventors: Matthew T. Moyer, Palmyra, PA (US); Kayla Hartz, University Park, PA (US); Courtney Lester, Hershey, PA (US)

(21) Appl. No.: 18/268,400

(22) PCT Filed: Dec. 21, 2021
(86) PCT No.: PCT/US21/64560

§ 371 Date: Jun. 20, 2023

Related U.S. Application Data
(60) Provisional application No. 63/128,713, filed on Dec. 21, 2020.

(51) Int. Cl. A61B 18/06; A61M 5/145; A61M 5/168; A61M 5/315; A61M 5/32

(52) U.S. Cl. CPC A61B 18/06; A61M 5/1452; A61M 5/16813; A61M 5/31581; A61M 5/32; A61B 2018/00577

(57) ABSTRACT

A fluid drug delivery system that includes an actuator connected to a valve for releasing a pressurized treatment fluid. A fluid chamber can be included that is configured to include the pressurized treatment fluid and include a volume that changes in response to the release of the pressurized treatment fluid. An external pressure interface can be in fluid communication with the fluid chamber. A pressure sensor-display dial can be included operatively connected to the fluid chamber and the external pressure interface to monitor fluid pressure of the fluid chamber and direct the operator to the correct pressure application.

### CROSS REFERENCE TO RELATED APPLICATION

This application claims the benefit of and priority to U.S. Provisional Patent Application No. 63/128,713 filed on 21 Dec. 2020. The entire contents of the application is incorporated herein by reference in their entirety.

### FIELD

The present disclosure relates to an apparatus and method for delivering fluid, such as treatment solutions to a patient.

### BACKGROUND

Pancreatic cancer is one of the most lethal malignancies and has a dismal five-year survival rate of approximately 10%. At least 20% of pancreatic malignancies stem from progression of mucinous-type pancreatic cysts, specifically mucinous cystic neoplasms (MCN) and intraductal papillary mucinous neoplasms (IPMN). Until recently, the only widely accepted approaches to managing mucinous pancreatic cysts were indefinite radiographic surveillance or major surgery, both which have significant limitations. Endoscopic ultrasound (EUS)-guided chemoablation is an innovative, minimally invasive, and rapidly evolving technique that can be utilized for treatment of appropriately selected, mucinous (precancerous) cysts, but there is currently no standardized tool available to treating gastroenterologist to provide reliable and safe infusion of the chemoablation agent. Current syringe systems are assembled using equipment designed for other indications and fail to adequately deliver viscous and therapeutic drug fluids. In particular, current infusion syringe set-ups often to leak at the valve connector, flanges bend or break under the pressure of pushing the plunger, and the pressure being applied is unknown. In addition, the syringe is not locked in and can escape the housing unless a make-ship mechanism is used such as tape or Velcro. This mechanism of holding the syringe in place within the housing is unreliable and impairs visualization of the volume of drug fluid remaining in the chamber. The solution of this disclosure resolves these and other problems in the art.

### SUMMARY

In some examples, a fluid drug delivery system is disclosed that includes an actuator connected to a valve for releasing a pressurized treatment fluid. A fluid chamber can be included that is configured to include the pressurized treatment fluid and include a volume that changes in response to the release of the pressurized treatment fluid. An external pressure interface can be in fluid communication with the fluid chamber. A pressure sensor can be included operatively connected to the fluid chamber and the external pressure interface to monitor fluid pressure of the fluid chamber and direct the operator to the correct pressure application.

In some examples, the actuator includes a reinforced flange.

In some examples, the system is hand-held.

In some examples, the system includes a display on the external pressure interface that displays the information relating to pressure sensed by the pressure sensor.

In some examples, the measurement taken by the sensor is pressure of the pressurized treatment fluid.

In some examples, the pressurized treatment fluid includes gemcitabine.

In some examples, the pressurized treatment fluid includes paclitaxel.

In some examples, the pressurized treatment fluid consists only of paclitaxel.

In some examples, the pressurized treatment fluid is delivered into a target cystic tumor through a needle (e.g., 19G needle, 22G needle, etc.) on a distal end of a tubular members (e.g., a fine needle aspiration needle) distally connected to the fluid delivery system at a predetermined fluid pressure of 4 ATMs for approximately 90 seconds, the pressurized treatment fluid including at least one or a combination of approximately 10 cm³ of gemcitabine and paclitaxel.

In some examples, the pressurized treatment fluid is delivered to a target cystic lesion through a needle on a distal end of one or more tubular members distally connected to the fluid delivery system at a predetermined fluid pressure of approximately 5 ATMs for approximately 76 seconds, the pressurized treatment fluid including at least one or a combination of approximately 10 cm³ of gemcitabine and paclitaxel.

In some examples, the pressurized treatment fluid is delivered to a target cystic lesion through a needle on the distal end of one or more tubular members distally connected to the fluid delivery system at a predetermined fluid pressure of approximately 6 ATMs for approximately 59 seconds, the pressurized treatment fluid including at least one or a combination of approximately 10 cm³ of gemcitabine and paclitaxel.

In some examples, the pressurized treatment fluid is delivered through a needle on a distal end of one or more tubular members distally connected to the fluid delivery system at a predetermined fluid pressure of approximately 8 ATMs for approximately 42 seconds, the pressurized treatment fluid including at least one or a combination of approximately 10 cm³ of gemcitabine and paclitaxel.

In some examples, the pressurized treatment fluid is delivered through a needle on a distal end of one or more tubular members distally connected to the fluid delivery system at a predetermined fluid pressure of approximately 10 ATMs for approximately 32 seconds, the pressurized treatment fluid including at least one or a combination of approximately 10 cm³ of gemcitabine and paclitaxel.

In some examples, the pressurized treatment fluid is delivered through a needle on a distal end of one or more tubular members distally connected to the fluid delivery system at a predetermined fluid pressure of approximately 6 ATMs for approximately 625 seconds, the pressurized treatment fluid including at least one or a combination of approximately 10 cm³ of gemcitabine and paclitaxel.

In some examples, the pressurized treatment fluid is delivered through a needle on a distal end of one or more tubular members distally connected to the fluid delivery system at a predetermined fluid pressure of 12 ATMs for approximately 26 seconds, the pressurized treatment fluid consisting of approximately 10 cm³ of paclitaxel.

In some examples, the pressurized treatment fluid is delivered through a needle on a distal end of one or more tubular members distally connected to the fluid delivery system at a predetermined fluid pressure of 12 ATMs for approximately 196 seconds, the pressurized treatment fluid consisting of approximately 10 cm³ of paclitaxel.

In some examples, the pressurized treatment fluid is delivered through a needle on a distal end of one or more tubular members distally connected to the fluid delivery system at a predetermined fluid pressure of 14 ATMs for approximately 21 seconds, the pressurized treatment fluid consisting of approximately 10 cm³ of paclitaxel.

In some examples, the pressurized treatment fluid is delivered through a needle on a distal end of one or more tubular members distally connected to the fluid delivery system at a predetermined fluid pressure of 14 ATMs for approximately 156 seconds, the pressurized treatment fluid consisting of approximately 10 cm³ of paclitaxel.

In some examples, the system is assembled with a proximal handle assembly comprising a trigger that when moved can cause the actuator to move proximally or distally to pressurized and/or cause the pressurized treatment fluid to be released.

In some examples, a fluid delivery system is disclosed that includes a pneumatic assembly including a pressurized treatment fluid. An actuator can be included with a flange button surface on a plunger rod and piston for pressurizing and/or releasing the pressurized treatment fluid from the pneumatic assembly. A hydraulic assembly can be distally connected to and in fluid communication with the pneumatic assembly, the hydraulic assembly configured to input and output the pressurized treatment fluid through an external interface in response to pressure from the pressurized treatment fluid. A pressure sensor can be operably connected to the hydraulic assembly to monitor pressure from the pneumatic assembly. A pressure interface can be connected to the sensor to display pressure measurements monitored by the sensor and setting a predetermined pressure for infusion.

In some examples, the pressurized treatment fluid includes gemcitabine.

In some examples, the pressurized treatment fluid includes paclitaxel.

In some examples, the pressurized treatment fluid consists only of paclitaxel.

In some examples, the hydraulic assembly includes a coupling separating the fluid chamber from the released pressurized treatment fluid.

In some examples, the coupling is configured to move within the hydraulic assembly while simultaneously maintaining a seal between the fluid chamber and the released pressurized treatment fluid.

In some examples, a method is disclosed for activating and/or infusing a chemoablation agent. The method includes actuating, by an actuator of any of the herein claimed or described fluid delivery systems, to pressurize a treatment fluid associated with the chemotherapeutic agent; measuring, by a pressure sensor in communication with the pneumatic assembly of the fluid delivery system, the pressure of the pressurized treatment fluid; and releasing the pressurized treatment fluid from the fluid delivery system to a hydraulic assembly in response to the pressurized treatment fluid achieving a predetermined pressure.

In some examples, the method includes delivering the pressurized treatment fluid into the center of a target pancreatic cystic tumor through a needle on a distal end of one or more tubular members distally connected to the fluid delivery system at a predetermined fluid pressure of approximately 4 ATMs for approximately 90 seconds, the pressurized treatment fluid including at least one or a combination of approximately 10 cm³ of gemcitabine and paclitaxel.

In some examples, the method includes delivering the pressurized treatment fluid into the center of a target pancreatic cystic tumor through a needle on a distal end of one or more tubular members distally connected to the fluid delivery system at a predetermined fluid pressure of approximately 5 ATMs for approximately 76 seconds, the pressurized treatment fluid including at least one or a combination of approximately 10 cm³ of gemcitabine and paclitaxel.

In some examples, the method includes delivering the pressurized treatment fluid through a needle on a distal end of one or more tubular members distally connected to the fluid delivery system at a predetermined fluid pressure of approximately 6 ATMs for approximately 59 seconds, the pressurized treatment fluid including at least one or a combination of approximately 10 cm³ of gemcitabine and paclitaxel. The needle can include a 19-gauge needle.

In some examples, the method includes delivering the pressurized treatment fluid through a needle on a distal end of one or more tubular members distally connected to the fluid delivery system at a predetermined fluid pressure of approximately 8 ATMs for approximately 42 seconds, the pressurized treatment fluid including at least one or a combination of approximately 10 cm³ of gemcitabine and paclitaxel.

In some examples, the method includes delivering the pressurized treatment fluid through a needle on a distal end of one or more tubular members distally connected to the fluid delivery system at a predetermined fluid pressure of approximately 10 ATMs for approximately 32 seconds, the pressurized treatment fluid including at least one or a combination of approximately 10 cm³ of gemcitabine and paclitaxel.

In some examples, the method includes delivering the pressurized treatment fluid through a needle on a distal end of one or more tubular members distally connected to the fluid delivery system at a predetermined fluid pressure of approximately 6 ATMs for approximately 625 seconds, the pressurized treatment fluid including at least one or a combination of approximately 10 cm³ of gemcitabine and paclitaxel. The needle can include a 22-gauge needle.

In some examples, the method includes delivering the pressurized treatment fluid through a needle on a distal end of one or more tubular members distally connected to the fluid delivery system at a predetermined fluid pressure of 12 ATMs for approximately 26 seconds, the pressurized treatment fluid consisting of approximately 10 cm³ of paclitaxel. The needle can include a 19-gauge needle.

In some examples, the method includes delivering the pressurized treatment fluid through a needle on a distal end of one or more tubular members distally connected to the fluid delivery system at a predetermined fluid pressure of 12 ATMs for approximately 196 seconds, the pressurized treatment fluid consisting of approximately 10 cm³ of paclitaxel. The needle can include a 22-gauge needle.

In some examples, the method includes delivering the pressurized treatment fluid through a needle on a distal end of one or more tubular members distally connected to the fluid delivery system at a predetermined fluid pressure of 14 ATMs for approximately 21 seconds, the pressurized treatment fluid consisting of approximately 10 cm³ of paclitaxel. The needle can include a 19-gauge fine needle aspiration (FNA) needle.

In some examples, the method includes delivering the pressurized treatment fluid through a needle on a distal end of one or more tubular members distally connected to the fluid delivery system at a predetermined fluid pressure of 14 ATMs for approximately 156 seconds, the pressurized treatment fluid consisting of approximately 10 cm³ of paclitaxel. The needle can include a 22-gauge FNA needle.

In some examples, the pressurized treatment fluid of the method is delivered to treat a renal neoplastic lesion.

In some examples, the pressurized treatment fluid of the method is delivered to treat appropriate symptomatic or neoplastic hepatic cysts or tumors.

In some examples, the method includes assembling the fluid delivery system with a handheld actuator system.

In some examples, the method includes moving a coupling between the hydraulic system and pneumatic assembly, within a hydraulic cylinder, while simultaneously maintaining a seal between the pneumatic assembly and the released pressurized treatment fluid.

To the accomplishment of the foregoing and related ends, certain illustrative aspects are described herein in connection with the following description and the appended drawings. These aspects are indicative, however, of but a few of the various ways in which the principles of the claimed subject matter can be employed and the claimed subject matter is intended to include all such aspects and their equivalents. Other advantages and novel features can become apparent from the following detailed description when considered in conjunction with the drawings.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1 is a side view of an example fluid drug delivery system of this disclosure fitting within a housing, in accordance with an exemplary embodiment of the present invention.

FIG. 2 is a side view of an example fluid drug delivery system, in accordance with an exemplary embodiment of the present invention.

FIG. 3A is a top perspective view of an example fluid drug delivery system illustrating an example pressure gauge, in accordance with an exemplary embodiment of the present invention.

FIG. 3B is a perspective view of an example fluid drug delivery system illustrating an example continuous valve, in accordance with an exemplary embodiment of the present invention.

FIG. 4 is a side perspective view of an example fluid drug delivery system, in accordance with an exemplary embodiment of the present invention.

FIG. 5 is an upper perspective view of an example fluid drug delivery system, in accordance with an exemplary embodiment of the present invention.

FIG. 6 is a close-up view of section A of FIG. 5.

FIG. 7A shows a table illustrating example pressures associated with duration of drug delivery with needles used with the systems of this disclosure in an example study.

FIG. 7B shows a table illustrating example pressures associated with duration of drug delivery with needles used with the systems of this disclosure in an example study.

FIG. 7C shows a table illustrating example pressures and corresponding characteristics observed in the system during an example study.

FIG. 8 depicts a graphical overview of one method 800 according to this disclosure.

### DETAILED DESCRIPTION

Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

It must also be noted that, as used in the specification and the appended claims, the singular forms “a,” “an” and “the” include plural referents unless the context clearly dictates otherwise. By “comprising” or “containing” or “including” it is meant that at least the named compound, element, particle, or method step is present in the composition or article or method, but does not exclude the presence of other compounds, materials, particles, method steps, even if the other such compounds, material, particles, method steps have the same function as what is named.

As used herein, the terms “about” or “approximately” for any numerical values or ranges indicate a suitable dimensional tolerance that allows the part or collection of components to function for its intended purpose as described herein. More specifically, “about” or “approximately” can refer to the range of values ±10% of the recited value, e.g. “about 90%” can refer to the range of values from 81% to 99%. In addition, as used herein, the terms “patient,” “host,” “user,” and “subject” refer to any human or animal subject and are not intended to limit the systems or methods to human use, although use of the subject invention in a human patient represents a preferred embodiment.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method can be performed in a different order than those described herein without departing from the scope of the disclosed technology. Similarly, it is also to be understood that the mention of one or more components in a device or system does not preclude the presence of additional components or intervening components between those components expressly identified.

As discussed herein, vasculature of a “subject” or “patient” can be vasculature of a human or any animal. It should be appreciated that an animal can be a variety of any applicable type, including, but not limited thereto, mammal, veterinarian animal, livestock animal or pet type animal, etc. As an example, the animal can be a laboratory animal specifically selected to have certain characteristics similar to a human (e.g., rat, dog, pig, monkey, or the like). It should be appreciated that the subject can be any applicable human patient, for example.

As discussed herein, “operator” can include a doctor, surgeon, medical professional, or any other individual or delivery instrumentation associated with using the system and its instrumentalities of this disclosure.

In a preferred aspect, the solution of this disclosure is not a method for treatment of the human or animal body by surgery or therapy and is not a diagnostic method practiced on the human or animal body. For example, when the solution involves clinically improving at least one clinical attribute during use, the clinical attribute may not be related to a method for treatment of the human or animal body by surgery or therapy or a diagnostic method practiced on the human or animal body.

The present disclosure is directed to a system configured to treat mucinous-type (precancerous) pancreatic cysts, specifically mucinous cystic neoplasms (MCN) and intraductal papillary mucinous neoplasms (IPMN) which are common and lead to approximately 20% of pancreatic cancer. In particular, the system of this disclosure is configured as an infusion system for efficiently and safely delivering chemotherapeutic agents under pressure, through an EUS-fine needle aspiration (FNA) needle, and across the wall of the esophagus, stomach or duodenum and delivering it into a tumor of interest. However, the disclosure is not so limited and the system of this disclosure can also be configured for use with hepatic lesions and renal tumors, solid hepatic or pancreatic mass lesions (e.g., ones in non-operative candidates).

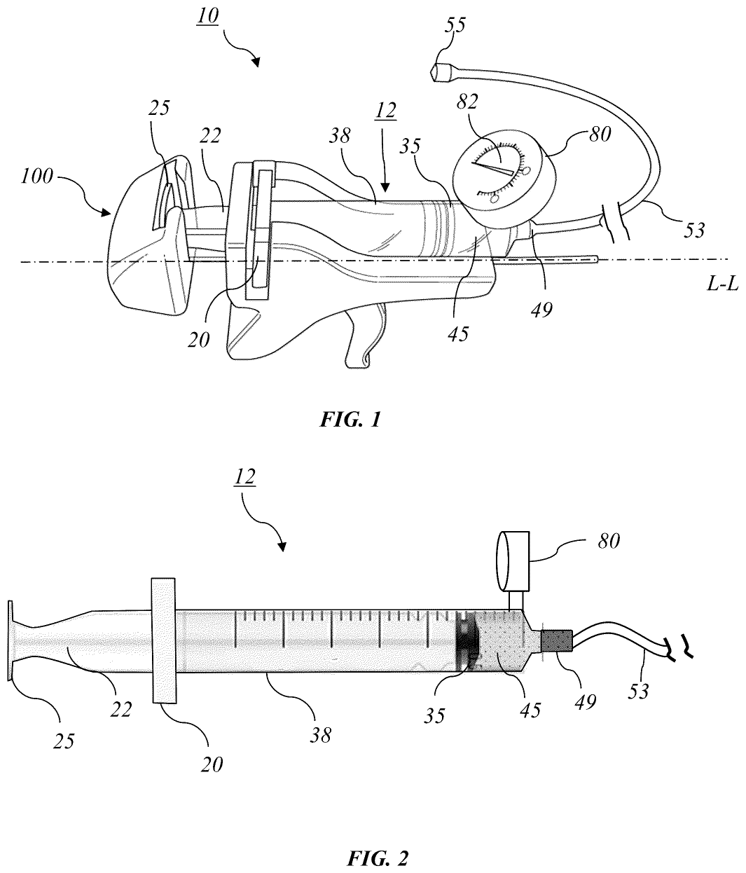

FIG. 1 is a side view of an example fluid drug delivery system 10 for delivering a viscous material. System 10 can be handheld and comprise an actuator 12 secured within a housing 100, as described in more detail herein. Actuator 12 can include a reinforced flange 20 positioned on a proximal end of a pressurized fluid chamber 38 and a rod 22 configured to tightly fit within fluid chamber 38. Rod 22 can include a plunger 35 positioned on a distal end and a proximal button 25 positioned on a proximal end. Proximal button 25 can be configured to receive an applied force to drive rod 22 further into the pressurized fluid chamber 38 and cause a fluid drug 45 to be delivered through a valve coupler 49 and a catheter lumen 53 to the patient. Actuator 12 can also include a pressure gauge 80 in fluid communication with fluid drug 45 so that a corresponding pressure setting is externally observed by an end-user prior to and/or during use. Pressure gauge 80 may have a display 82 for providing information relating to pressure sensed by a pressure sensor operatively connected to fluid chamber 38.

Fluid chamber 38 can be of any suitable dimension (length, width, and diameter) for containing the proper volume of drug 45 for a particular procedure. Preferably, fluid chamber 38 can hold between 2 and 20 cc of viscous treatment fluid. Fluid chamber 38 can be constructed from polycarbonate and be PVC free so as to be compatible with drug 45 when it includes gemcitabine and/or paclitaxel. In some examples, material for the construction of fluid chamber 38 can include polyether ether ketone (PEEK), nylon, polypropylenes, and polytetraflouroethylenes (PTFE) or other fluorinated polycarbons could also be used. Any suitable materials, which are durable, sterilizable, biofriendly, chemically compatible with viscous materials and substantially reinforced under the expected operating pressures of up to 20 ATM, but could, also withstand pressures up to about 50 ATM (e.g., up to 45 ATM, up to 40 ATM, up to 35 ATM, up to 30 ATM, up to 25 ATM, up to 20 ATM, up to 15 ATM, up to 10 ATM, and any value between, e.g., up to 32 ATM).

In some examples, reinforced flange 20 positioned on the proximal end of chamber 38 can be reinforced with one or more thickened members to ensure material integrity and prevent snapping during use. Flange 20 can be constructed from polycarbonate and be PVC free so as to be compatible with drug 45 when it includes gemcitabine and/or paclitaxel. In some embodiments, flange 20 can be made of disposable materials such that system 10 can be disposed of. Alternatively, or in addition thereto, flange 20 can be reinforced with other materials such as metals, carbon composites, glass additives, or other suitable additives to strengthen flange 20 without altering the size.

Any fluid drug 45 is contemplated for use with the system of this disclosure including but not limited to a chemoablation agent such as gemcitabine, paclitaxel, or an infusion admixture of gemcitabine with paclitaxel. Additional drugs can be used in system 10, particularly viscous fluids that require controlled delivery.

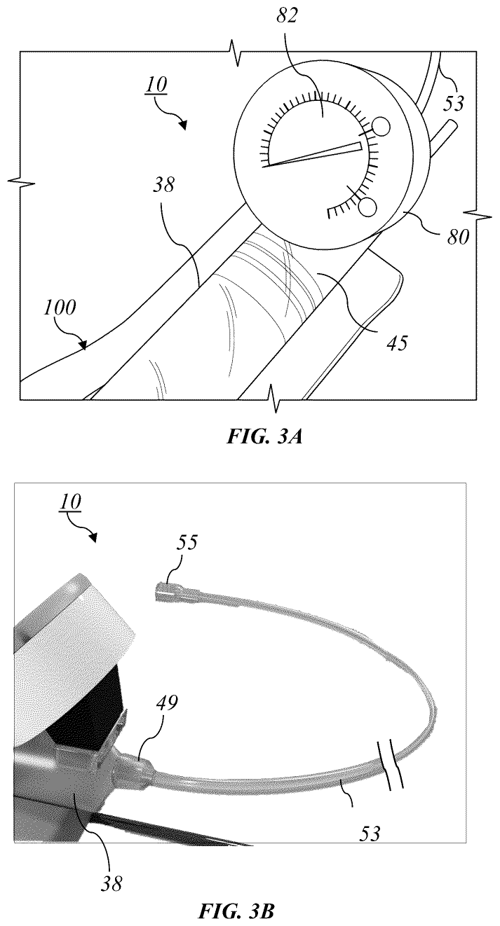

Instead of a luer lock to couple the syringe to the catheter tubing, valve coupler 49 can be made from the same material as fluid chamber 38 such that the joint is continuous and prevents leaking of the viscous drug 45 under high pressure. Valve coupler 49 can have a diameter less than fluid chamber 38. In addition, valve coupler 49 can have a cross-sectional shape matching that of fluid chamber 38 or can be of any suitable shape to withstand the force applied to the system 10. As shown in more detail in FIG. 3B, valve coupler 49 can be shaped like a funnel and include a catheter tubing 53 also made from the same or similar material as fluid chamber 38.

Housing 100 can be manufactured specially to secure actuator 12. In particular, housing 100 can include a channel for receiving fluid chamber 38 of actuator 12, a first slot perpendicular to the channel for receiving reinforced flange 20 of actuator 12, and a second slot perpendicular to the channel for receiving proximal button 25 of actuator 12. The second slot can be separate from the channel and operatively connected to the channel to apply pressure to actuator 12, thereby releasing the pressurized treatment fluid from system 10. In general, housing 100 can include a mechanism that causes the proximal button 25 and rod 22 of actuator 12 to move. In some embodiments, housing 100 can include a trigger and/or a threaded rod that, when moved along a longitudinal axis L-L, can cause the second slot and proximal button 25 of actuator 12 to compress or expand along longitudinal axis L-L. Movement of housing 100 second slot while securing actuator 12 applies a force to drive rod 22 of actuator 12 further into pressurized fluid chamber 38. Although not shown in FIG. 1, housing 100 can further include a strap or other suitable locking mechanism to hold actuator 12 within the channel and the first and second slots. Housing 100 can be made of varying sizes to securely fit actuator 12 of varying dimensions. In some embodiments, multiple actuators 10 can be used in system 10. For instance, actuator 12 may be filled with the viscous drug 45 and interchanged by the operator as one is emptied. In other embodiments, the system 10 may be a one-use only device. In addition, or alternatively thereto, housing 100 can be a pre-existing housing capable of securely fitting actuator 12. For example, housing 100 can be the Alliance™ II Inflation Handle (Boston Scientific, Marlborough, MA), the Quantum™ Biliary Inflation Device (Cook Medical, Bloomington, IN) and other suitable housings for fitting an actuator or syringe.

A person skilled in the art will appreciate that other actuators can be used in system 10. Examples of linear actuators include a rack and pinion, ratchet and pawl, an electric motor with a worm gear, an angled clutch plate on a rod, or a walking beam. An example of a nonlinear actuator that could also be used is a hydraulic pump.

FIG. 2 is a perspective view of actuator 12 comprising reinforced flange 20, pressurized fluid chamber 38, rod 22, pressure gauge 80, valve coupler 49 with catheter lumen 53 for continuous flow of the fluid drug 45 through the actuator. Rod 22 comprises plunger 35 and proximal button 25. Actuator 12 can comprise fluid drug 45.

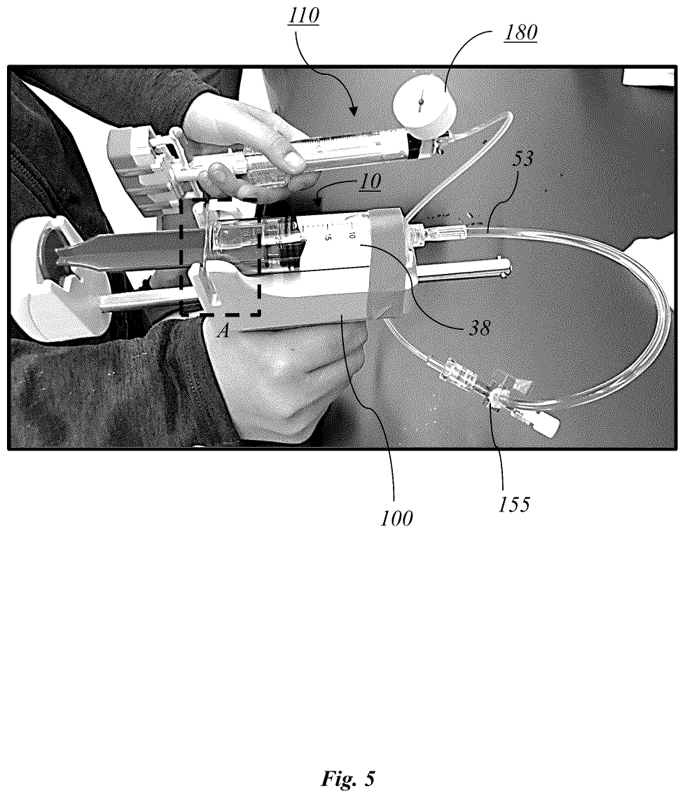

FIG. 3A is a top perspective view of system 10 showing pressure gauge 80 and display 82. In some examples, pressure gauge 80 may be attachable to fluid chamber 38, housing 100, or detachable (i.e., to be held separately from system 10) as shown in FIG. 5. Pressure gauge can be in fluid communication with fluid drug 45 so that a corresponding pressure setting is externally observed by an end-user prior to and/or during use. Gauge display 82 can be user friendly and include one or more pressure indicators that indicate to an end-user a desired pressure setting associated with fluid drug 45 contained in chamber 38 and to be delivered via catheter 53 to a patient. As shown in FIG. 3A, gauge display 82 can display the information relating to pressure sensed by a pressure sensor associated with chamber 38. For instance, at 25 ATM with a 19-gauge needle, the delivery of drug is within an applied force range safe for maintaining system 10, but at 40 ATM or above with a 19-gauge needle, the system 10 may begin to experience mechanical failure. As would be appreciated by one of skill in the relevant art, the pressure resistance for delivering viscous fluid increases by a factor based on the diameter of the needle being used and also on the length of tubing the fluid will travel through the catheter. In turn, monitoring fluid pressure of chamber 38 in real-time can be extremely informative to direct the operator to initiate and/or adjust drug delivery at the correct pressure application.

FIG. 3B is a perspective view of system 10 showing valve coupler 49 connected to fluid chamber 38. Valve coupler 49 can be positioned at a distal end of chamber 38 through which fluid drug 45 can be delivered through catheter or fluid lumen 53. In some examples, valve couple 49 can be a luer-lock fitting or luer A-barb fitting to prevent leakage of the drug 45 from the connector. Alternatively, fluid chamber 38 and valve couple 49 can be of a single piece and/or material such that there no joint between fluid chamber 38 and catheter 53. Catheter 53 and any corresponding couplers can be high-pressure PVC-free connector tubing. A standard endoscopic ultrasound FNA needle fitting 55 can be positioned at a distal end of catheter 53 to receive an FNA needle (e.g., 19g, 22g, etc.) for delivering fluid drug from system 10 to the patient using EUS.

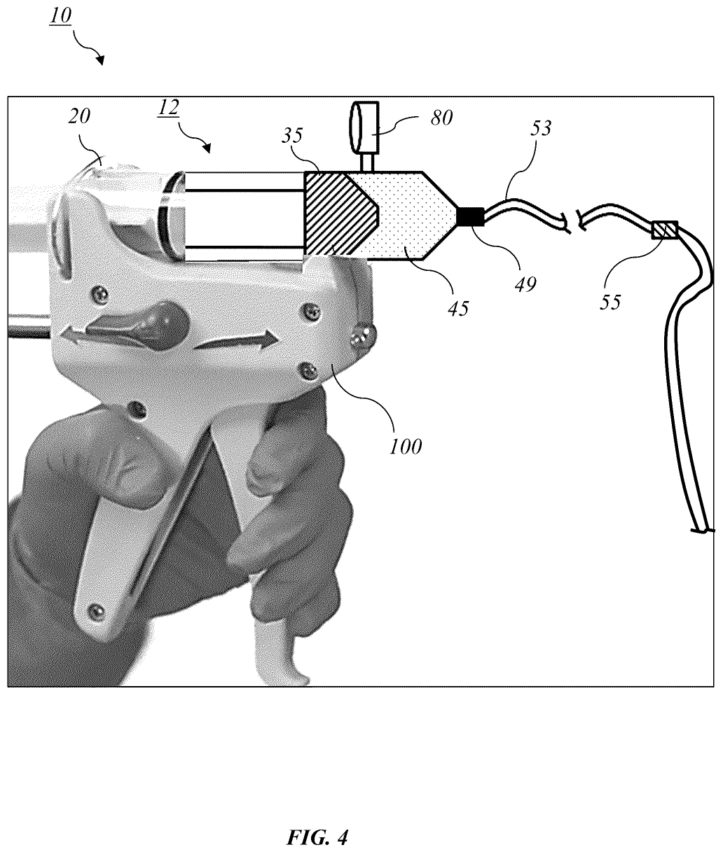

FIG. 4 is a perspective view of system 10 assembled within a hand-operated actuator housing 100 comprising a trigger. As can be seen, actuator 12 is assembled about its fluid chamber 38 and its flange 20 aligned so that that actuating the trigger grip of housing 100 causes plunger 35 and rod 22 to pressurize and cause fluid drug 45 to be delivered from system 10 and egress through valve coupler 49 into catheter 53.

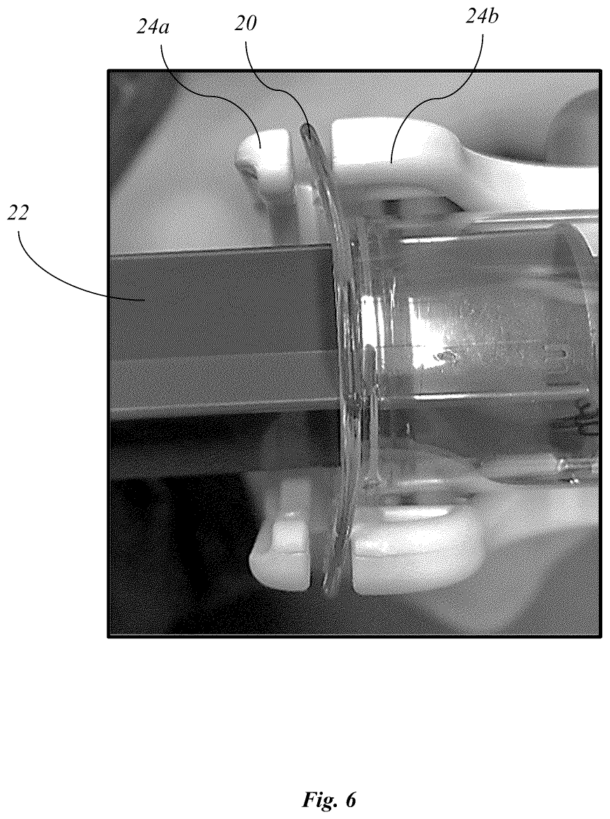

This disclosure is more clearly understood with a corresponding study discussed more particularly below. System 10 was slightly modified for the example study of this disclosure to accommodate a modified pressure gauge 180 so as to monitor pressure and fail settings during pressurized drug delivery. FIG. 6 is a close-up of section A of FIG. 5 more clearly showing flange 20 when positioned between slots 24a, 24b as rod 22 is translated into and out of chamber 38. In some examples, pressure gauge 80 can comprise an external pressure interface, a pressure sensor, and a display that provides information relating to the pressure detected by the pressure sensor. In addition, or alternatively thereto, the pressure of system 10 can be monitored by a separate pressure gauge 180 that is connected to and in fluid communication with system 10 via a hydraulic assembly 110. Hydraulic assembly 110 can be configured to input and output fluid drug 45 through the external interface in response to pressure from the pressurized treatment fluid. The pressure sensor can be connected to hydraulic assembly 110 to monitor pressure within catheter 53 and from system 10. In addition, hydraulic assembly 110 can include a coupling 155 separating fluid chamber 38 from the released fluid drug 45. Coupling 155 can be configured to move within hydraulic assembly 110 while simultaneously maintaining a seal between fluid chamber 38 and the released fluid drug 45.

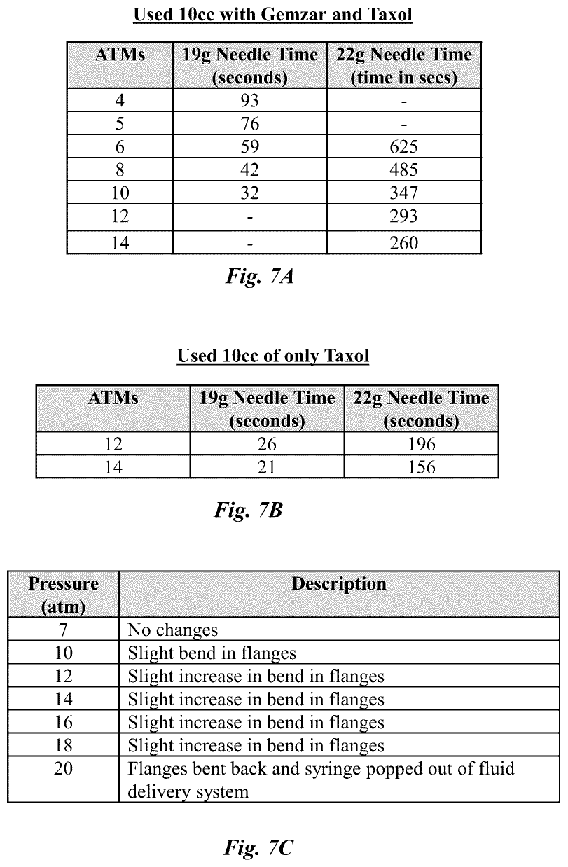

As for the study itself, system 10 of this disclosure was evaluated for drug infusion times using both 19 and 22-gauge needles. In the study, fluid drug included both gemcitabine+paclitaxel and paclitaxel alone, as well as atmospheres it takes for the fluid drug delivery system to fail at high-pressures. In particular, FIG. 7A shows a table illustrating example pressures associated with duration of drug delivery with both 19 and 22-gauge needles during delivery of 10 cc of gemcitabine and paclitaxel. FIG. 7B shows a table illustrating example pressures associated with duration of drug delivery with both 19 and 22-gauge needles during delivery of 10 cc of only paclitaxel. FIG. 7C shows a table illustrating example pressures and corresponding characteristics observed in the system during an example study. In the study, it was revealed that the 22-gauge needle takes much longer to infuse than the 19-gauge needle. It was unexpected that Taxol alone takes a shorter time to infuse using the system of this disclosure than the combination of gemcitabine and paclitaxel. The flanges of the system were also significant with the syringe and evidenced the clear need for the reinforced flanges of the system of this disclosure.

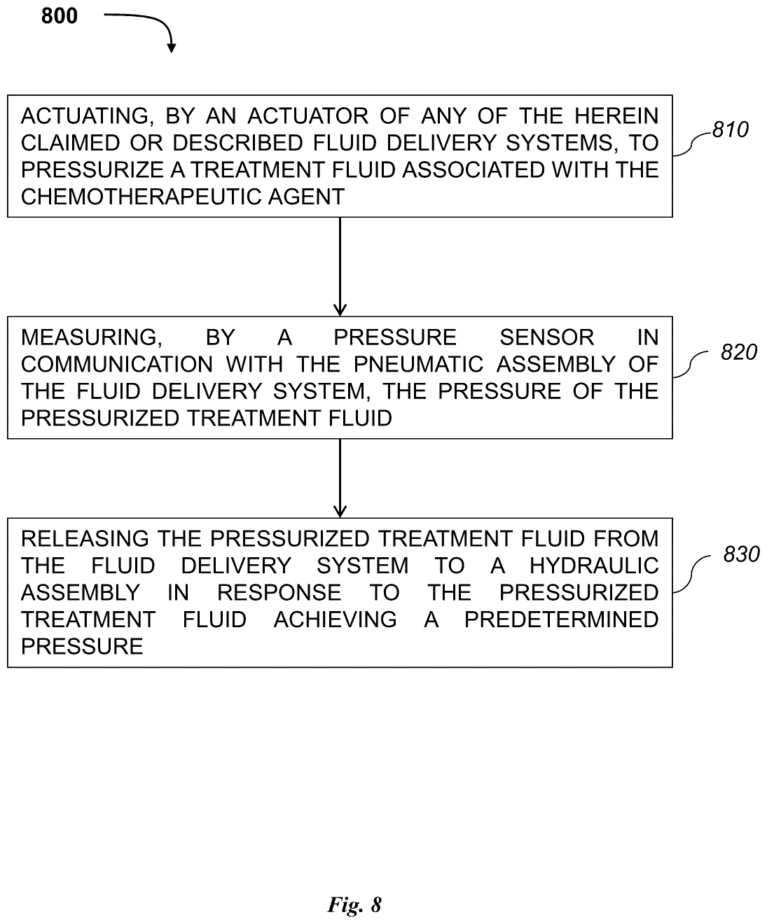

FIG. 8 depicts a graphical overview of one method 800 according to this disclosure. The method 800 can include 810 actuating, by an actuator of any of the herein claimed or described fluid delivery systems, to pressurize a treatment fluid associated with the chemotherapeutic agent; 820 measuring, by a pressure sensor in communication with the pneumatic assembly of the fluid delivery system, the pressure of the pressurized treatment fluid; and 830 releasing the pressurized treatment fluid from the fluid delivery system to a hydraulic assembly in response to the pressurized treatment fluid achieving a predetermined pressure.

In some examples, method 800 can include delivering the pressurized treatment fluid into the center of a target pancreatic cystic tumor through a needle on a distal end of one or more tubular members distally connected to the fluid delivery system at a predetermined fluid pressure of 4 ATMs for approximately 90 seconds, the pressurized treatment fluid comprising at least one or a combination of approximately 10 cm³ of gemcitabine and paclitaxel.

In some examples, method 800 can include delivering the pressurized treatment fluid into the center of a target pancreatic cystic tumor through a needle on a distal end of one or more tubular members distally connected to the fluid delivery system at a predetermined fluid pressure of approximately 5 ATMs for approximately 76 seconds, the pressurized treatment fluid comprising at least one or a combination of approximately 10 cm³ of gemcitabine and paclitaxel.

In some examples, method 800 can include delivering the pressurized treatment fluid through a needle on a distal end of one or more tubular members distally connected to the fluid delivery system at a predetermined fluid pressure of approximately 6 ATMs for approximately 59 seconds, the pressurized treatment fluid comprising at least one or a combination of approximately 10 cm³ of gemcitabine and paclitaxel.

In some examples, method 800 can include delivering the pressurized treatment fluid through a needle on a distal end of one or more tubular members distally connected to the fluid delivery system at a predetermined fluid pressure of approximately 8 ATMs for approximately 42 seconds, the pressurized treatment fluid comprising at least one or a combination of approximately 10 cm³ of gemcitabine and paclitaxel.

In some examples, method 800 can include delivering the pressurized treatment fluid through a needle on a distal end of one or more tubular members distally connected to the fluid delivery system at a predetermined fluid pressure of approximately 10 ATMs for approximately 32 seconds, the pressurized treatment fluid comprising at least one or a combination of approximately 10 cm³ of gemcitabine and paclitaxel.

In some examples, method 800 can include delivering the pressurized treatment fluid through a needle on a distal end of one or more tubular members distally connected to the fluid delivery system at a predetermined fluid pressure of approximately 6 ATMs for approximately 625 seconds, the pressurized treatment fluid comprising at least one or a combination of approximately 10 cm³ of gemcitabine and paclitaxel.

In some examples, method 800 can include delivering the pressurized treatment fluid through a needle on a distal end of one or more tubular members distally connected to the fluid delivery system at a predetermined fluid pressure of 12 ATMs for approximately 26 seconds, the pressurized treatment fluid consisting of approximately 10 cm³ of paclitaxel.

In some examples, method 800 can include delivering the pressurized treatment fluid through a needle on a distal end of one or more tubular members distally connected to the fluid delivery system at a predetermined fluid pressure of 12 ATMs for approximately 196 seconds, the pressurized treatment fluid consisting of approximately 10 cm³ of paclitaxel.

In some examples, method 800 can include delivering the pressurized treatment fluid through a needle on a distal end of one or more tubular members distally connected to the fluid delivery system at a predetermined fluid pressure of 14 ATMs for approximately 21 seconds, the pressurized treatment fluid consisting of approximately 10 cm³ of paclitaxel.

In some examples, method 800 can include delivering the pressurized treatment fluid through a needle on a distal end of one or more tubular members distally connected to the fluid delivery system at a predetermined fluid pressure of 14 ATMs for approximately 156 seconds, the pressurized treatment fluid consisting of approximately 10 cm³ of paclitaxel.

The specific configurations, choice of materials and the size and shape of various elements can be varied according to particular design specifications or constraints requiring a system or method constructed according to the principles of the disclosed technology. Such changes are intended to be embraced within the scope of the disclosed technology. The presently disclosed embodiments, therefore, are considered in all respects to be illustrative and not restrictive. It will therefore be apparent from the foregoing that while particular forms of the disclosure have been illustrated and described, various modifications can be made without departing from the spirit and scope of the disclosure and all changes that come within the meaning and range of equivalents thereof are intended to be embraced therein.

1. A fluid drug delivery system, comprising:
an actuator connected to a valve for releasing a pressurized treatment fluid;
a fluid chamber configured to comprise the pressurized treatment fluid and comprising a volume that changes in response to the release of the pressurized treatment fluid;
an external pressure interface in fluid communication with the fluid chamber; and
a pressure sensor operatively connected to the fluid chamber and the external pressure interface, configured to monitor fluid pressure of the fluid chamber and direct an operator of the system to a correct pressure application.

2. The system of claim 1, the actuator comprising a reinforced flange.

3.-5. (canceled)

6. The system of claim 1, wherein the pressurized treatment fluid comprises gemcitabine, paclitaxel, or combinations thereof.

7. (canceled)

8. (canceled)

9. The system of claim 1, wherein the pressurized treatment fluid is delivered into a target cystic tumor through a needle on a distal end of a tubular member distally connected to the fluid delivery system at a predetermined fluid pressure of 4 ATMs for approximately 90 seconds, the pressurized treatment fluid comprising at least one or a combination of approximately 10 cm³ of gemcitabine and paclitaxel.

10. The system of claim 1, wherein the pressurized treatment fluid is delivered to a target cystic lesion through a needle on a distal end of one or more tubular members distally connected to the fluid delivery system at a predetermined fluid pressure of approximately 5 ATMs for approximately 76 seconds, the pressurized treatment fluid comprising at least one or a combination of approximately 10 cm³ of gemcitabine and paclitaxel.

11. The system of claim 1, wherein the pressurized treatment fluid is delivered to a target cystic lesion through a needle on a distal end of one or more tubular members distally connected to the fluid delivery system at a predetermined fluid pressure of approximately 6 ATMs for approximately 59 seconds, the pressurized treatment fluid comprising at least one or a combination of approximately 10 cm³ of gemcitabine and paclitaxel.

12. The system of claim 1, wherein the pressurized treatment fluid is delivered through a needle on a distal end of one or more tubular members distally connected to the fluid delivery system at a predetermined fluid pressure of approximately 8 ATMs for approximately 42 seconds, the pressurized treatment fluid comprising at least one or a combination of approximately 10 cm³ of gemcitabine and paclitaxel.

13. The system of claim 1, wherein the pressurized treatment fluid is delivered through a needle on a distal end of one or more tubular members distally connected to the fluid delivery system at a predetermined fluid pressure of approximately 10 ATMs for approximately 32 seconds, the pressurized treatment fluid comprising at least one or a combination of approximately 10 cm³ of gemcitabine and paclitaxel.

14. The system of claim 1, wherein the pressurized treatment fluid is delivered through a needle on a distal end of one or more tubular members distally connected to the fluid delivery system at a predetermined fluid pressure of approximately 6 ATMs for approximately 625 seconds, the pressurized treatment fluid comprising at least one or a combination of approximately 10 cm³ of gemcitabine and paclitaxel.

15. The system of claim 1, wherein the pressurized treatment fluid is delivered through a needle on a distal end of one or more tubular members distally connected to the fluid delivery system at a predetermined fluid pressure of 12 ATMs for approximately 26 seconds, the pressurized treatment fluid consisting of approximately 10 cm³ of paclitaxel.

16. The system of claim 1, wherein the pressurized treatment fluid is delivered through a needle on a distal end of one or more tubular members distally connected to the fluid delivery system at a predetermined fluid pressure of 12 ATMs for approximately 196 seconds, the pressurized treatment fluid consisting of approximately 10 cm³ of paclitaxel.

17. The system of claim 1, wherein the pressurized treatment fluid is delivered through a needle on a distal end of one or more tubular members distally connected to the fluid delivery system at a predetermined fluid pressure of 14 ATMs for approximately 21 seconds, the pressurized treatment fluid consisting of approximately 10 cm³ of paclitaxel.

18. The system of claim 1, wherein the pressurized treatment fluid is delivered through a needle on a distal end of one or more tubular members distally connected to the fluid delivery system at a predetermined fluid pressure of 14 ATMs for approximately 156 seconds, the pressurized treatment fluid consisting of approximately 10 cm³ of paclitaxel.

19. (canceled)

20. A fluid delivery system, comprising:
a pneumatic assembly comprising a pressurized treatment fluid;
an actuator comprising a flange button surface on a plunger rod and piston for pressurizing and/or releasing the pressurized treatment fluid from the pneumatic assembly;
a hydraulic assembly distally connected to and in fluid communication with the pneumatic assembly, the hydraulic assembly configured to input and output the pressurized treatment fluid through an external interface in response to pressure from the pressurized treatment fluid;
a pressure sensor operably connected to the hydraulic assembly to monitor pressure from the pneumatic assembly; and
a pressure interface connected to the sensor to display pressure measurements monitored by the sensor and setting a predetermined pressure for infusion.

21. The system of claim 20, wherein the pressurized treatment fluid comprises gemcitabine, paclitaxel, or combinations thereof.

22. (canceled)

23. (canceled)

24. The system of claim 20, wherein the hydraulic assembly comprises a coupling separating a fluid chamber within the pneumatic assembly from the released pressurized treatment fluid, and configured to move within the hydraulic assembly while simultaneously maintaining a seal between the fluid chamber and the released pressurized treatment fluid.

25. (canceled)

26. A method of activating and/or infusing a chemoablation agent, comprising:
actuating, by an actuator of any of the herein claimed or described fluid delivery systems, to pressurize a treatment fluid associated with a chemotherapeutic agent;
measuring, by a pressure sensor in communication with the pneumatic assembly of the fluid delivery system, a pressure of a pressurized treatment fluid; and
releasing the pressurized treatment fluid from the fluid delivery system to a hydraulic assembly in response to the pressurized treatment fluid achieving a predetermined pressure.

27. The method according to claim 26, further comprising:
delivering the pressurized treatment fluid into a center of a target pancreatic cystic tumor through a 19-gauge needle on a distal end of one or more tubular members distally connected to the fluid delivery system at a predetermined fluid pressure ranging from approximately 4 ATMs to approximately 10 ATMs for a time period ranging from approximately 32 seconds to approximately 93 seconds, the pressurized treatment fluid comprising at least one or a combination of approximately 10 cm³ of gemcitabine and paclitaxel.

28.-32. (canceled)

33. The method according to claim 26, further comprising:
delivering the pressurized treatment fluid through a 22-gauge needle on a distal end of one or more tubular members distally connected to the fluid delivery system at a predetermined fluid pressure ranging from approximately 6 ATMs to approximately 14 ATMs for a time period ranging from approximately 260 seconds to approximately 625 seconds, the pressurized treatment fluid comprising at least one or a combination of approximately 10 cm³ of gemcitabine and paclitaxel.

34.-42. (canceled)

43. The method according to claim 26, wherein the pressurized treatment fluid is delivered to treat a renal neoplastic lesion, neoplastic hepatic cysts, tumors, or combinations thereof.

44.-46. (canceled)
